(19) United States
(12) Patent Application Publication
(10) Pub. No.: US 2003/0049262 A1
(43) Pub. Date: Mar. 13, 2003

(54) Methods for identification, diagnosis, and treatment of breast cancer

(76) Inventors: Susan Love, Pacific Palisades, CA (US); Julian Nikolchev, Portola Valley, CA (US); David Hung, Belmont, CA (US); Hui Cen, Oakland, CA (US)

Correspondence Address: BANNER & WITCOFF, Ltd., 1001 G Street, N.W. Eleventh Floor, Washington, DC, 20001-4597, US

(21) Appl. No.: 09/565,642

(22) Filed: May 5, 2000

Related U.S. Application Data
(63) Continuation-in-part of application No. 09/410,336, filed on Oct. 1, 1999.
(60) Provisional application No. 60/102,829, filed on Oct. 2, 1998.

(51) Int. Cl. G01N 33/574; A61K 39/395; C12Q 1/68; C07H 21/04

(52) U.S. Cl. 424/155.1; 435/6; 435/7.23

(57) ABSTRACT

The disclosure describes methods of identifying and treating premalignant and malignant breast cancer, and systems and kits for the same. The diagnostic methods comprise intraductal administration of agents that can clear in the body comprising a targeting molecule either alone acting as an identifying agent, or coupled to an identifying agent; the therapeutic methods comprise intraductal administration of agents that can clear n the body comprising a targeting molecule coupled to a therapeutic agent or a targeting molecule having also therapeutic activity.

### CROSS-REFERENCES TO RELATED APPLICATIONS

This application is a continuation-in-part of application Ser. No. 09/410,336, filed on Oct. 1, 1999, which claimed the benefit of provisional patent application Ser. No. 60/102,829, filed on Oct. 2, 1998, under 37 CFR §1.78(3), the full disclosures of which are incorporated herein by reference.

### BACKGROUND OF THE INVENTION

1. Field of the Invention

The present invention relates generally to medical methods for identifying, diagnosing and treating breast cancer.

Breast cancer is the most common cancer in women, with well over 100,000 new cases being diagnosed each year. In the United States, one out of every eight women will eventually be diagnosed with breast cancer. Although many treatments have been developed over the years, effective treatment still relies largely on early detection of the disease. Even greater numbers of women, however, have symptoms associated with breast diseases, both benign and malignant, and must undergo further diagnosis and evaluation in order to determine whether breast cancer exists. To that end, a variety of diagnostic techniques have been developed, the most common of which are surgical techniques including core biopsy and excisional biopsy. Recently, fine needle aspiration (FNA) cytology has been developed which is less invasive than the surgical techniques, but which is not always a substitute for surgical biopsy.

A variety of other diagnostic techniques have been proposed for research purposes. Of particular interest to the present invention, fluids from the breast ducts have been externally collected, analyzed, and correlated to some extent with the risk of breast cancer. Such fluid collection, however, is generally taken from the surface of the nipple and includes material from all of the ductal structures. Information on the condition of an individual duct is generally not provided. Information on individual ducts can be obtained through cannulation and endoscopic or fluoroscopic examination, but such examinations have been primarily in women with nipple discharge or for research purposes and have generally not examined each individual duct in the breast.

Since breast cancer usually arises from a single ductal system and exists in a precancerous state for a number of years, endoscopy in and fluid collection from individual breast ducts holds great diagnostic promise for the identification of intermediate markers. Of particular interest to the present invention, it would be of great value to be able to reliably collect ductal fluids and cellular and non-cellular marker materials (e.g., epithelial and other cells as well as proteins, carbohydrates, and other non-cellular marker materials) from the individual breast ducts on a duct-by-duct basis. By examining the collected marker materials, cancerous and precancerous conditions within each duct could be identified at a very early stage. Moreover, by associating the condition with a specific duct, treatment could be directed specifically at that duct in an attempt to enhance the effectiveness of the treatment and minimize trauma to the patient.

The ability to perform such diagnostic techniques, however, has been limited. Heretofore, it has been very difficult to identify ductal orifices in a reliable and consistent manner. That problem, however, has been addressed by the invention reported in co-pending U.S. application Ser. No. 08/931,786, filed on Sep. 16, 1997, the full disclosure of which is incorporated herein by reference. By labeling the ductal orifices, the location of the entry orifice for each duct can be established.

Even though access to all of the ducts in a breast can now be achieved, successful diagnostic methods will depend on the ability to collect cellular and non-cellular materials from at least, most, and preferably all, regions of each ductal network. Breast ducts have highly complex and convoluted three-dimensional geometries, with more remote portions of the network having increasingly smaller diameters. Thus, obtaining representative material samples from throughout a ductal network represents a significant challenge.

Prior attempts to obtain cellular material from individual breast ducts have been only partly successful. As reported by one of the inventors herein, in Love and Barsky (1996) The Lancet 348: 997-999, breast ducts have been cannulated with a rigid cannula and instilled with very small volumes (0.2 ml to 0.5 ml) of saline. Saline was recovered separately through a capillary tube, and cellular material recovered from the saline. It was not clear, however, if cellular material was recovered from most or all portions of the ductal network. Unless such representative samples can be obtained, reliable diagnostics cannot be performed. While the paper proposes development of a two-lumen catheter, no such catheter or its use is described in the publication.

Breast cancer usually begins in the cells lining a breast duct and in the terminal ductal lobular unit, with the first stage thought to be excessive proliferation of individual cell(s) leading to “ductal hyperplasia.” Some of the hyperplastic cells may then become atypical, with a significant risk of the atypical hyperplastic cells becoming neoplastic or cancerous. Initially, the cancerous cells remain in the breast ducts, and the condition is referred to as ductal carcinoma in situ (DCIS). After a time, however, the cancerous cells are able to invade outside of the ductal environment, presenting the risk of metastases which can be fatal to the patient. Breast cancer proceeds through discrete premalignant and malignant cellular stages: normal ductal epithelium, atypical ductal hyperplasia, ductal carcinoma in situ (DCIS), and finally invasive ductal carcinoma. The first three stages are confined within the ductal system and, therefore, if diagnosed and treated, lead to the greatest probability of cure.

While breast cancer through the DCIS phase is in theory quite treatable, effective treatment requires both early diagnosis and an effective treatment modality. At present, mammography is the state-of-the-art diagnostic tool for detecting breast cancer. Often, however, mammography is only able to detect tumors that have reached a size in the range from 0.1 cm to 1 cm. Such a tumor mass may be reached as long as from 8 to 10 years following initiation of the disease process. Detection of breast cancer at such a late stage is often too late to permit effective treatment.

Alternative diagnostic modalities which promise much earlier detection of breast cancer and DCIS are described in co-pending U.S. application Ser. Nos. 08/931,786, 09/067,661, 09/301,058, and 60/122,076 the full disclosures of which are incorporated herein by reference. Together, these applications describe techniques for identifying one or more (usually all) individual ductal orifices on a nipple in a breast and for collecting cellular and other materials from individual ductal networks to determine if hyperplasia, DCIS, or other abnormal conditions are present in that network. While these techniques will be very useful in providing early and accurate diagnosis of breast cancer and other disease conditions, they do not directly provide for prevention and treatment of the condition once it is diagnosed.

Conventional treatments for breast cancer have been focused on the treatment of a latter stage disease and include removal of the breast, localized removal of the tumor (“lumpectomy”), radiation, and chemotherapy. While these techniques are often very effective, they suffer from certain deficiencies. Removal of the breast provides the best assurance against local recurrence of the cancer, but is disfiguring and requires the patient to make a very difficult choice. Lumpectomy is less disfiguring, but is associated with greater risk of recurrence of the cancer. Radiation and chemotherapy are arduous and are not completely effective against recurrence. Such conventional treatments will not always be able to take full advantage of emerging diagnostic techniques which promise to allow detection of precancerous and cancerous conditions in the breast at a very early stage.

A method for treating and/or inhibiting cancer and other abnormal conditions in the ductal linings of the breast is described in co-pending U.S. application Ser. No. 09/313,463, filed on Sep. 17, 1999, the full disclosure of which is incorporated herein by reference. In that application, radiofrequency and other forms of energy are used to necrose the ductal lining to inhibit hyperplasia growth. While believed to be effective, it is not clear whether these techniques will be sufficient to treat all cancers and other ductal abnormalities.

It would be desirable to provide improved and alternative techniques for identifying, diagnosing, treating, and/or preventing breast cancer and invasive carcinoma, and precancerous conditions such as ductal carcinoma in situ (DCIS), and atypical ductal hyperplasia (ADH). In particular, it would be desirable to provide treatment modalities that can be used in conjunction with techniques which provide early diagnosis of DCIS and other abnormal conditions within individual breast ducts. Such techniques should be less invasive and traumatic to the patient than the present techniques, should result in minimum or no disfigurement of the breast, and should be effective locally within target sites within the breast duct and/or throughout an entire ductal network and terminal ductal lobular unit. Preferably, the techniques should be capable of being performed in a single or very few treatment session(s). At least some of these objectives will be met by the invention described hereinafter.

2. Description of the Background Art

Co-pending U.S. application Ser. Nos. 08/931,786 and 09/067,661, 09/313,463, and 09/301,058 have been described above and are hereby incorporated herein in their entireties. Publications by one of the inventors herein relating to breast duct access include Love and Barsky (1996) Lancet 348: 997-999; Love (1992) “Breast duct endoscopy: a pilot study of a potential technique for evaluating intraductal disease,” presented at 15th Annual San Antonio Breast Cancer Symposium, San Antonio, Tex., Abstract 197; Barsky and Love (1996) “Pathological analysis of breast duct endoscoped mastectomies,” Laboratory Investigation, Modern Pathology, Abstract 67. A description of the inventor's earlier breast duct access work was presented in Lewis (1997) Biophotonics International, pages 27-28, May/June 1997.

Nipple aspiration and/or the introduction of contrast medium into breast ducts prior to imaging are described in Sartorius (1995) Breast Cancer Res. Treat. 35: 255-266; Satorious et al., (1977) “Contrast ductography for the recognition and localization of benign and malignant breast lesions: An improved technique,” in: Logan (ed.), Breast Carcinoma, New York, Wiley, pp. 281-300; Petrakis (1993) Cancer Epidem. Biomarker Prev. 2: 3-10; Petrakis (1993) Epidem. Rev. 15: 188-195; Petrakis (1986) Breast Cancer Res. Treat. 8: 7-19; Wrensch et al., (1992) Am. J Epidem. 135: 130-141; Wrensch et al., (1990) Breast Cancer Res. Treat. 15: 39-51; and Wrensch et al., (1989) Cancer Res. 49: 2168-2174. The presence of abnormal biomarkers in fine needle breast aspirates is described in Fabian et al., (1993) Proc. Ann. Meet. Am. Assoc. Cancer Res. 34: A1556. The use of a rigid 1.2 mm ductoscope to identify intraductal papillomas in women with nipple discharge is described in Makita et al., (1991) Breast Cancer Res. Treat. 18: 179-188. The use of a 0.4 mm flexible scope to investigate nipple discharge is described in Okazaki et al., (1991) Jpn. J Clin. Oncol. 21: 188-193. The detection of CEA in fluids obtained by a nipple blot is described in Imayama et al., (1996) Cancer 78: 1229-1234. Delivery of epithelium-destroying agents to breasts by ductal cannulation is described in WO 97/05898 and U.S. Pat. No. 5,763,415.

Energy-mediated ablation of the uterus, gall bladder, blood vessels, and other hollow body organs are described in the following U.S. Pat. Nos.: 4,776,349; 4,869,248; 4,872,458; 4,979,948; 5,045,056; 5,100,388; 5,159,925; 5,222,938; 5,277,201; 5,242,390; 5,403,311; 5,433,708; 5,507,744; and 5,709,224.

Treating breast cancer by intraductal administration of a cytotoxic agent or an epithelial destroying agent is described in WO 97/05898.

### SUMMARY OF THE INVENTION

The present invention provides improved methods, systems, and kits for identification, diagnosis (including staging), and treatment of malignant and premalignant lesions of the breast. In particular, the improved methods and apparatus analyze, diagnose and stage the cells or fluids found in breast duct and provide for treating cancerous cells or tissues and/or for preventing the occurrence of cancerous cell growth. These methods will be performed in patients at risk of cancer or other diseases of the breast ducts.

Premalignant and malignant lesions are usually confined to the breast ductal system and the terminal ductal lobular unit. The terminal ductal lobular unit or TDLU is the network of ducts and ductal tributaries located at and towards the base of the breast. This network flows into the milk ducts of the breast that extend from the TDLU towards the nipple. Ultimately, the milk ducts each end at a ductal orifice located on the nipple surface. Women have an average of 6 to 12 ductal orifices on each nipple. For description and definition of terminal ductal lobular unit see Wellings SR, Pathol Res Pract 166(4): 515-35 (1980), Stirling and Chandler, Virchows Arch A Pathol Anat Histol 372(3): 205-26 (1976), and Fraser et al., Am J Surg Pathol 22(12): 1521-7 (1998).

Access, diagnosis and treatment of breast cancer according to the present invention are directed at individual ducts, ductal networks, and terminal ductal lobular unit within the breast. Accessing the lesions within the duct, prior to the lesion invading surrounding tissues, provides a far more sensitive and accurate method of screening for and localizing neoplastic breast lesions than currently available techniques such as physical exam, mammography, magnetic resonance imaging (MRI) and impedance mapping. Thus, methods of the present invention permit identification of which individual duct or ducts with a breast display premalignant and/or malignant lesions. Optionally, the methods further permit localization of the lesion(s) within an individual duct.

In addition to identifying the ductal networks which display premalignant and malignant lesions and precisely defining the disease location within the ductal network(s), the invention provides novel methods for staging a neoplastic breast lesion and a means to identify peripheral (sentinel) lymph node involvement. Lymph node involvement includes sentinel node involvement. The sentinel node is defined as the first-line axillary lymphatic drainage node in breast cancer (see Salmon and Fried, Presse Med 27(11): 509-12 (1998)). The peripheral lymph nodes of the breast include mostly axillary nodes and to a lesser extent parastemal nodes (see Bland and Copeland, The Breast: Comprehensive Management of Benign and Malignant Diseases 1991 W. B. Saunders Co., Philadelphia, Pa. pages 30-31). Thus, the invention provides a means to identify whether the tumor or lesion has spread to the sentinel lymph node. See also Bland and Copeland, The Breast: Comprehensive Management of Benign and Malignant Diseases 1991 W. B. Saunders Co., Philadelphia, Pa. pages 27-29, 342, and 737-738.

The ability to both pinpoint the location of the breast lesion(s) and to define the stage of the disease will greatly enhance the ability of the physician to decide upon and implement the most appropriate surgical or medical therapy, thereby leading to superior clinical outcomes. Furthermore, the increased sensitivity of the technique over current screening procedures and its ability to precisely localize breast lesion(s) allows identification of lesions at their earliest possible stages (before metastasis has occurred), thereby increasing the likelihood of cure by allowing precise curative surgical resections or specifically targeted medical therapies.

In a specific aspect of the present invention, targeting molecules are used to mediate the delivery of targeting agents, e.g., labeling moieties or substances, and/or therapeutic agents to the lesion. The targeting molecules can be antibodies, ligands, receptors, or the like, and will be capable of preferentially binding target substances in the lesion. Labeling moieties and substances which serve as the targeting agent may be conventional labels, such as radioactive labels, fluorescent labels, chemiluminescent labels, bioluminescent labels, and the like. Therapeutic agents can be anti-neoplastic drugs, toxins, antibodies (which may serve as both the targeting and therapeutic substances), and the like. The therapeutic agents will be locally delivered to inhibit, ablate, necrose, or otherwise treat the breast intraductal lesions.

Breast cancer proceeds through discrete premalignant and malignant cellular stages: normal ductal epithelium, atypical ductal hyperplasia, ductal carcinoma in situ, and finally invasive ductal carcinoma. The first three stages are confined within the ductal system, including the terminal ductal lobular unit, and therefore if diagnosed and treated, offer the greatest probability of cure. All of these stages can be characterized by unique cellular markers and epitopes, each of which can be targeted by specific molecules coupled to identifying agents to define the precise location of the lesions within the ductal system. Staging refers to staging of the ductal epithelial cells by identifying, e.g., whether the cells are normal, precancerous, or cancerous (e.g., whether they are benign, premalignant or malignant). Further detail can be added with the process of staging the ductal epithelial cells, e.g., precancerous cells can be identified as hyperplastic, atypically hyperplastic, or presenting low-grade ductal carcinoma in situ. Likewise, cancerous cells might be identified, e.g., as high-grade carcinoma in situ or invasive cancer.

Presently, the most useful stage for a surgeon to identify is carcinoma including carcinoma in situ and invasive carcinoma. Breast cancer presently is most likely identified by modalities that are the present standard of care including mammography and physical exam, and what is detected by these modalities is generally carcinoma (either in situ or invasive). Thus, the greatest aide to a surgeon vis-á-vis the present invention is localized identification of the lesion and/or tumor in the duct or ductal terminal lobular unit so that the surgeon may excise the cancerous tissue cleanly and completely during a surgery (e.g., a “Y” or “J” or other type of excision). The invention also provides a method of locating a lesion that can be detected by magnetic resonance imaging (MRI) or other such means that does not require the breast tissue to be opened, including also, e.g., positron emission tomography (PET). A targeting molecule labeled with and/or conjugated to an MRI-detectable molecule (e.g., those available from Pharmacyclics, Inc., Sunnyvale, Calif.) or opaque molecule, etc. or a radioactive compound such as e.g., iodine-125 or indium-111 or other such compounds disclosed in U.S. Pat. No. 4,938,948, the full disclosure of which is incorporated herein by reference) can provide additional or separate guidance to a surgeon before cutting tissue, or to aid in an MRI-assisted excisional biopsy. Thus, a preferred targeting molecule will identify, bind or detect carcinoma. Detecting atypical lesions in contrast will permit development of new treatments for the early stages of cancer and precancerous conditions. Additionally, it will permit identification of patients who require more careful monitoring and counseling.

The invention provides a method by which the targeting agent(s) coupled to identifying and/or therapeutic molecules are delivered directly through the nipple (usually through one or more of the ductal orifices) to the ductal network(s) through cannulation of specific ducts. Local delivery in this manner will enhance the effectiveness of the identifying agents by allowing increased concentrations of identifying agents to reach the target site than might be possible by systemic delivery. Local delivery in this manner may also enhance the effectiveness of therapeutic agents by allowing increased concentrations of therapeutic agents to reach the target site than might be possible by systemic delivery. For example, dosages that might be intolerable if delivered systemically could be delivered locally without unacceptable side effects and toxicity.

Local delivery also provides the opportunity to treat the patient with agents that can cross-react with other tissues and which would otherwise be eliminated from a systemic protocol (e.g., an agent that reacts with breast cancer tissue and with e.g., lung or liver tissue). Thus, many potential breast cancer or breast precancer therapeutic agents that would cross react with other tissues in the body if delivered systemically can be delivered locally to the breast without fear of cross-reaction with other tissues in the body.

The phrase “targeting agent” includes compounds or substances (such as antibodies, (including humanized, partially humanized, and non-human antibodies) proteins, peptides, polynucleotides, drugs, chemicals, ligands, receptors, etc.) that bind specifically to the target cell or target antigen (e.g., cell surface or secreted antigen) to become incorporated into or in some fashion serve as a vehicle for identification of cell types of interest. Targeting agents for the present invention can include agents specific for intraductal cellular targets such as Her-2 (EGF receptor) or ligands or receptors of the ErbB family, heat shock protein (HSP), such as heat shock protein 27 and the like; cytokeratins (particularly keratin 14); estrogen and progesterone receptors (or any androgen or other steroid receptor); cathepsins, including cathepsin-D; growth factors/cytokines including FGF1-18, VEGF, IGF-I, IGF-II, PDGF, KGF, EGF, PLGF, HGF, TNF, TGF alpha, TGF beta and the like; growth factor receptors to FGF1-18, VEGF, IGF-I, IGF-II, PDGF, KGF, EGF, PLGF, HGF, TNF, TGF alpha and beta and the like; urokinase, urokinase-type plasminogen activator (UPA), plasmin, antiplasmin, UPA receptor (UPAR), fibrinogen, PAI-1 and 2, -chemokines (both C-C and C-X-C); integrins, selectins, cadherins, including alpha v beta 3; CEA, PSA, maspin, fas, fas ligand; collagenases, metalloproteinases, TIMP's, disrupted basement membrane epitopes, stromolysin-3-Ki-67, Ki-S1, p53, nm23, bcl-2, p21 ras, cyclins, pS2. Also included are antibodies generated from any of the active agents listed herein. Other targeting agents can include small molecules, proteins/peptides, lipids, or nucleic acids. Certain antibodies chosen may themselves have both therapeutic as well as targeting capability. Such an example would include the monoclonal antibody to the Her-2 receptor as this is currently an approved therapy for breast cancer.

Thus, in some instances, the targeting agents may possess therapeutic activity. Because they are “targeting agents” they will preferentially bind lesion cells and display limited or preferably no binding to other epithelial and ductal lining cells.

The therapeutic activity of the targeting and/or therapeutic agents can be anything that disrupts, inhibits, retards, or eliminates the cancer or precancer cells target or other antigen from thriving and making more of the same cells. Targeting agents may also be conjugated to a therapeutic agent for targeting abnormal cells and delivering the conjugated therapeutic agent to the diseased or abnormal cells. The targeting agents themselves would not be considered cytotoxic agents, but rather the targeting agents specifically target and bind cancerous or precancerous cells and allow contact of the cancerous or precancerous cells with the cytotoxic agent that is conjugated to the therapeutic agent. Nonspecific binding and nonspecific cytotoxic activity is thereby avoided by avoiding contact between healthy cells and the cytotoxic agent. The targeting molecules acting in this capacity act to deliver an active therapeutic agent specifically to a cancerous or precancerous cell, and the active therapeutic agent (conjugated to the targeting agent) may include cytotoxic agents such as those listed, for example in WO 97/05898, and can also include other agents e.g., cytolytic agents, growth inhibiting agents, antagonists, agonists, and any other therapeutically active agents capable of being conjugated to a targeting molecule and delivered effectively to a cancerous or precancerous cell intraductally.

Thus, lipophilic drug-containing liposomes can be conjugated to a monoclonal antibody or other targeting molecule (e.g., a protein, peptide, nucleic acid, or small organic molecule) that specifically targets cancerous or precancerous cells and the conjugated compound can be delivered intraductally to therapeutically treat a breast cancer or precancer. The drug-containing liposomes can contain any therapeutically active drug desired, e.g., a cytotoxic agent (e.g., such as those cytotoxic agents as listed in WO 97/05898), or any other therapeutically active agent that can be carried and released by the liposomes upon contact of the targeting agent (to which the liposome is conjugated) with the cancerous or precancerous cell or associated antigen. The drug containing liposomes can be any available liposomes including those mentioned herein, and also including those described in U.S. Pat. No. 5,512,294.

Furthermore, the invention provides a method of identifying atypical or cancerous cells lining or proximal to the ductal networks using an identifying agent, for example, monoclonal antibodies or other molecules directed against overexpressed or stage-specific cellular epitopes or targets such as growth factors or their receptors, integrins, proteases, and tumor specific antigens and the like. Preferably, the identifying agent will be specific for a cell membrane bound target, but may also be able to detect other cellular components including, e.g., soluble protein products produced from the cells and present in proximity to the parent cell, and intracellular products using an identifying agent capable of penetrating the cell wall, for example an intrabody, or cell wall permeable peptide or small molecule. The identifying agent may include small chemical entities, proteins, or nucleic acids which will be imageable themselves or which will be coupled to identifying compounds such as radio-opaque, radioactive or similarly detectable substances (see also the substances described in U.S. Pat. No. 4,938,948). Alternatively, the primary lesion-targeting agent may itself serve as a target for a secondary antibody or molecule that carries or is itself an identifying compound. The identification, localization, and delineation of the extent of the intraductal lesion(s) greatly enhance the ability of physicians to localize and direct appropriate therapies to the lesion(s), for example “Y” or “J” type of surgical excisions.

The phrase “identifying agent” includes antibodies, liposomes filled with imaging compounds (usually coupled to an antibody or other targeting molecule), fluorescent compounds, radioactive compounds, radiolucent compounds and the like, that serve as an aid to visualization through an imaging process. The identifying agent may already be coupled to a targeting agent (such as an antibody or other targeting molecule) or may require a secondary targeting agent for specific localization to the site of interest. Alternatively, the identifying agent may in and of itself be capable of binding a targeting agent thereby providing identification through visualization. Specific identifying agents include:—gadolinium (all radiographic contrast agents)—technitium (all radionuclides used in nuclear medicine imaging); ferromagnetic material (detectable by a magnetic sensor)—sonographically reflective material (detected with ultrasound); electrically conductive material (detected and mapped with electronic sensors)—thermographically reflective material (detected thermographically)—impedance-altering molecule which can be detected on impedance breast mapping any other agent that is externally monitorable or visualizable. The targeting agent may also be found in a carrier including liposomes, immunoliposomes, branched polymers; proteins or any macromolecule and the like.

An alternative approach that increases the specificity of identifying agents involves taking advantage of fibrinolytic enzymes or proteases at lesion sites that are used to cleave substrates that “light up” areas of increased fibrinolytic or protease activity. For example, increased UPAR, UPA, cathepsin, collagenase, or metalloproteinase expression levels in DCIS or invasive cancer might be used to pinpoint these lesions within the duct with an identifying agent activated by these enzymes.

These targeting agents optionally may be coupled to a wide variety of identifying agents. Ideally, the identifying agent should be of very high specific activity and amplifiable (i.e., akin to “branched DNA” in concept) to maximize ease of detection. Some potential identifying agents are listed below.

The invention also provides a method of grading or staging the invasiveness or seriousness of cancerous or precancerous growth using selected cancer cell markers. The expression of two or more markers associated with various stages of cancer invasiveness can be simultaneously intraductally measured using the monoclonal antibodies or other labeling agents described above. As a specific example, Her-2 expression appears to increase dramatically in DCIS and carries on at elevated levels even after progression to invasive cancer. Stromolysin-3 on the other hand appears to be highly expressed only in cells adjacent to an invasive cancer. If antibodies to Her-2 and stromolysin-3 are coupled to different identifying agents, the presence of one or the other or both aids the physician in more precisely determining the stage of the neoplastic lesion. Thus, the use of different markers such as these may allows for more accurate staging of neoplastic lesions of the milk duct and provides a non-invasive alternative for the physicians to determine the most appropriate therapies for the treatment of these lesions.

The phrase “cancer cell markers” refers to all molecules, molecular structures, and/or other epitopic or antigenic surface or other features which are characteristic of neoplastic cells, particularly of the ductal epithelial cells. Exemplary marker molecules are listed elsewhere in this application. The invention further provides a method of determining lymph node involvement. Diffusible dyes or radionuclides are intraductally administered and targeted specifically to intraductal lesions. Such agents identify key sentinel nodes more accurately than currently available surgical methods or other invasive, intra- or peri-tumorally injected agents, or even intraductally administered but not lesion-targeted markers. An advantage of this approach is the focused release of the agent in the vicinity of the lesion rather than throughout the entire ductal network. This allows the more precise identification of the lymph nodes most likely to drain a particular lesion. Thus, the invention provides a level of tumor or lesion staging previously unattainable without an invasive or surgical procedure.

The invention provides a method of treating premalignant or malignant breast cancer, said method comprising providing a targeting molecule coupled to a therapeutic agent; and delivering the coupled compound through a preselected individual breast duct in an amount sufficient to inhibit proliferation of the cancerous cells. The invention also provides a method of treating a premalignant or malignant breast cancer, said method comprising providing a targeting molecule itself having therapeutic activity; and delivering the coupled compound through a preselected individual breast duct in an amount sufficient to inhibit proliferation of the cancerous cells. The therapeutic method can include that the premalignant or malignant breast cancer comprises cells having a stage selected from the group consisting of hyperplasia, atypical hyperplasia, low-grade ductal carcinoma in situ, high-grade ductal carcinoma in situ, and invasive carcinoma.

The invention further provides a method by which the targeting agents as described above may be coupled to a variety of therapeutic agents or serve as the target for a primary or secondary antibody-coupled agent or other molecule capable of delivering localized therapy to a lesion or the entire ductal network as needed. Targeting agents of high valency are desirable because they are be able to simultaneously carry large quantities of both diagnostic identifying agents and therapeutic molecules to enhance their diagnostic sensitivity and therapeutic capability. These agents are then administered directly into the ductal network, which greatly enhances the diagnostic and therapeutic capability of these molecules.

The phrase “therapeutically active agents” refers to any biologically active agent capable of achieving a desired therapeutic effect, such as killing or inhibiting proliferation of a neoplastic cell. Exemplary bioactive therapeutically active agents include proteins, carbohydrates, nucleic acids, small organic molecules, specifically including e.g., enzymes, antibiotics, anti-neoplastic agents, bacterio static agents, bacteriosidle agents, anti-viral agents, hemostatic agents, anti-inflammatory agents, hormones, anti-angiogenic agents, antibodies, and the like. Preferred therapeutically active agents for use in the present invention include chemotherapeutic small molecules (i.e., cyclophosphamide, adriamycin, tamoxifen, raloxifene, taxol, etc); therapeutic proteins (i.e., herceptin, maspin, angiostatin, endostatin, etc.); and genes or nucleic acids (p53, maspin, ribozymes).

These therapeutic agents optionally may be coupled to a wide variety of active agents or alternatively carriers, like liposomes or immunoliposomes as defined above.

The invention also provides an alternative method of identifying cells at the site of a cancerous lesion. Cells undergoing division at abnormally high rates may be targeted for identifying and or therapy. A number of established agents can be preferentially taken up by proliferating cells within or proximal to the milk duct(s). These agents might include: Nucleoside analogs (BrdU, labeled thymidine and the likes) or cellular components related to increased protein, lipid or nucleic acid synthesis and requirements.

The present invention provides also that a preferred agent for imaging or therapy targeted to a lesion in a duct is an agent that is readily cleared by the body without requiring removal of the unbound agent from the duct. It is generally believed that removal of unbound agent from the duct would require an additional access step into the duct to wash the duct with fluid in order to collect the unbound material in the fluid and retrieve the fluid solution from the duct through, or example, the lumen of the tool used to access the duct. Thus, a preferred agent whether targeting a lesion for imaging or for therapeutic purposes is an agent that clears in the body safely within a reasonable period of time of the infusion into the duct.

It is a discovery of the present invention that certain antibodies and fragments thereof are optimal for delivery of imaging or therapeutic agents because they are cleared by the body and do not require removal of the unbound antibody from the body by a practitioner. An advantage is provided to the procedure using these antibody and antibody fragments that can be safely and efficiently cleared by the body because the fewer times that the practitioner needs to access and/or wash the duct the better. It is a further discovery of the present invention that certain small molecules or other targeting agents may possess the ability both to target the cells of a lesion, but also that the unbound moieties are small enough to clear in the body without needing to be washed out of the duct or removed from the duct (or these moieties possess other qualities that permit clearance in the body). Size is presumed to play a role in the ability of any of these moieties that target cells of a lesion to clear through the duct and by the body without requiring that the unbound molecules be removed from the body e.g. through the duct in a lavage procedure. However, size may not be the only factor contributing to this ability, and the invention is not limited to theories of how it works.

Accordingly, there is provided by the invention a method of identifying the location of premalignant or malignant breast cancer within a mammalian body comprising a breast duct or breast ductal network by providing a targeting molecule coupled to an identifying agent; delivering the coupled compound through a preselected individual breast duct in an amount sufficient to identify premalignant or malignant cancerous cells; identifying any bound cells; and allowing any unbound coupled compound to clear in the body; wherein any coupled compound that does not bind a cell in the duct diffuses out of the breast ductal network and is absorbed and cleared in the body without requiring removal by a practitioner of the unbound coupled compound. Delivering can comprise cannulation or catheterization of the breast duct, for example. A procedure can comprise accessing and delivering the coupled compound to more than one duct on a breast. This could include up to all the ducts on a breast (e.g. from a total of about 6 to about 9 ducts). The cells can be identified for the purpose of excising tissue surrounding and including the cells. The targeting agent can comprise an agent selected from the group consisting of a protein, a polypeptide, a peptide, an antibody, an antibody fragment, a F(ab′) fragment of an antibody, a F(ab′)₂ fragment of an antibody, an Fc portion of an antibody, a heavy chain of an antibody, a light chain of an antibody, a humanized antibody, a humanized antibody fragment, a ligand, a receptor, a drug, a chemical, a lipid, a liposome, a small molecule, and a nucleic acid. Preferred agents are small enough to clear in the body and/or possess the ability to be cleared based on parameters other than size. Clearance of an agent in the body without requiring such procedures, for example, as removal of the agent (e.g. with a washing and fluid retrieval procedure) is desired in the procedure for imaging and treating a breast duct or ducts by the method of the invention. In addition to Fab′ fragments and other antibody fragments, preferred agents may be small molecules such as for example, sestamibi.

The invention also provides a method of identifying the location of premalignant or malignant breast cancer within a mammalian body comprising a breast duct or breast ductal network, said method comprising providing a identifying agent; delivering the identifying agent through a preselected individual breast duct in an amount sufficient to identify premalignant or malignant cancerous cells; identifying any bound cells; and allowing any unbound identifying agent to clear in the body; wherein any identifying agent that does not bind a cell in the duct diffuses out of the breast ductal network and is absorbed and cleared by the body without requiring removal by a practitioner of the unbound identifying agent. As with the targeting molecule, delivery of the identifying agent can comprise cannulation or catheterization of the breast duct. The identifying agent can be delivered to more than one duct on a breast. This could include up to all the ducts on a breast (e.g. from a total of about 6 to about 9 ducts). The cells are identified for the purpose of excising tissue surrounding and including the cells. The identifying agent can comprise an agent selected from the group consisting of a protein, a polypeptide, a peptide, an antibody, an antibody fragment, a F(ab′) fragment of an antibody, a F(ab′)₂ fragment of an antibody, an Fc portion of an antibody, a heavy chain of an antibody, a light chain of an antibody, a humanized antibody, a humanized antibody fragment, a ligand, a receptor, a drug, a chemical, a lipid, a liposome, a small molecule, and a nucleic acid. The small molecule can be, for example sestamibi.

The invention also provides treatment methods that are analogous to the imaging/targeting/identifying methods. Thus, a method of treating premalignant or malignant breast cancer located within a mammalian body is provided, said method comprising providing a targeting molecule coupled to a therapeutic agent; delivering the coupled compound through a preselected individual breast duct in an amount sufficient to inhibit proliferation of the cancerous cells; allowing unbound coupled compound to clear through the body; wherein any coupled compound that does not bind a cell in the duct diffuses out of the breast ductal network and is absorbed and cleared by the body without requiring removal by a practitioner of the unbound coupled compound. Once again, delivering can comprise cannulation or catheterization of the breast duct. The coupled compound can be delivered to more than one duct on a breast, for example, where it is desirable to treat more than one duct. This could include up to all the ducts on a breast (e.g. from a total of about 6 to about 9 ducts). The targeting agent can comprise an agent selected from the group consisting of a protein, a polypeptide, a peptide, an antibody, an antibody fragment, a F(ab′) fragment of an antibody, a F(ab′)₂ fragment of an antibody, an Fc portion of an antibody, a heavy chain of an antibody, a light chain of an antibody, a humanized antibody, a humanized antibody fragment, a ligand, a receptor, a drug, a chemical, a lipid, a liposome, a small molecule, and a nucleic acid. The small molecule can be, for example, sestamibi. The therapeutic agent can be selected from the group consisting of a cytotoxic agent, a cytolytic agent, a cytostatic agent, a growth inhibiting agent, an antagonist, an agonist, and a drug or agent containing liposome. The therapeutic agent can comprise an agent with therapeutic activity against cancerous or precancerous cells that can be coupled to a targeting agent.

The invention also provides a treatment method using a targeting molecule itself having therapeutic activity. Thus, the invention provides a method of treating a premalignant or malignant breast cancer located within a mammalian body, said method comprising providing a targeting molecule itself having therapeutic activity; delivering the targeting molecule through a preselected individual breast duct in an amount sufficient to inhibit proliferation of the cancerous cells; allowing unbound targeting molecule to clear through the body; wherein any targeting molecule that does not bind a cell in the duct diffuses out of the breast ductal network and is absorbed and cleared by the body without requiring removal by a practitioner of the targeting molecule. Once again, delivering can comprise cannulation or catheterization of the breast duct; and the targeting molecule can be delivered to more than one duct on a breast (e.g. from about 6 to about 9 ducts in total). The targeting molecule can comprise an agent selected from the group consisting of a protein, a polypeptide, a peptide, an antibody, an antibody fragment, a F(ab′) fragment of an antibody, a F(ab′)₂ fragment of an antibody, an Fc portion of an antibody, a heavy chain of an antibody, a light chain of an antibody, a humanized antibody, a humanized antibody fragment, a ligand, a receptor, a drug, a chemical, a lipid, a liposome, a small molecule, and a nucleic acid. The small molecule can be for example sestamibi. The therapeutic activity can be selected from the group consisting of a cytotoxicity, a cytolytic activity, cytostatic activity, growth inhibition, antagonism, an agonism, and immunotoxicity. The therapeutic activity can be effective against cancerous or precancerous cells.

In cases of imaging, identifying, locating, or treating, the premalignant or malignant breast cancer can comprise cells having a stage selected from the group consisting of hyperplasia, atypical hyperplasia, low-grade ductal carcinoma in situ, high-grade ductal carcinoma in situ, and invasive carcinoma.

In accordance with the usefulness of the invention in a clinical context, the invention provides a kit for localizing or treating lesions in a breast duct, said kits comprising at least one catheter configured to access a ductal network in a human breast; and instructions for use setting forth a method according to any of those imaging, identifying, locating, or treating methods described above. The kit can further comprise at least one container holding a reagent which is used in the method being performed with the kit. The kit can further comprise a package holding the catheter and the instructions for use of the catheter with the other components of the kit. The kit can further comprise a reagent, wherein the reagent comprises an agent comprising at least a portion selected from the group consisting of a protein, a polypeptide, a peptide, an antibody, an antibody fragment, a F(ab′) fragment of an antibody, a F(ab′)₂ fragment of an antibody, an Fc portion of an antibody, a heavy chain of an antibody, a light chain of an antibody, a humanized antibody, a humanized antibody fragment, a ligand, a receptor, a drug, a chemical, a lipid, a liposome, a small molecule, and a nucleic acid.

### BRIEF DESCRIPTION OF THE DRAWINGS

FIG. 1 illustrates a kit comprising at least one ductal access cannula, optional reagents, instructions for use, and optional packaging for performing methods according to the present invention.

### DESCRIPTION OF THE SPECIFIC EMBODIMENTS

Targeting molecules can be used to identify and/or treat premalignant and malignant breast cancer lesions when the target molecule is administered locally. The targeting molecule may be selected based on the type of lesion and the specificity of the targeting molecule. For example, targeting molecules for a cancerous lesion would include targeting molecules specific for carcinoma cells or antigens. Likewise, targeting molecules for atypical cells would include molecules specific for atypical ductal epithelial cells or antigens. For example, antibodies to Her-2 antigen can detect carcinoma in situ, and thus antibody or other targeting molecules for Her-2 would be used for detecting in situ carcinoma. A surgeon wishing to identify a carcinoma in a breast duct for excision, would select an antibody specific for carcinoma, either in situ or invasive, for example. Thus, for example, humanized anti-c-erbB-2 antibodies (herceptin) can be used in localized treatment administered to the breast duct for treatment of cancer (e.g., invasive carcinoma) or precancer (e.g., low grade ductal carcinoma in situ) as described in Luftner et al., Int J Biol Markers 14(2): 55-9 (1999). Other targeting molecules that can act therapeutically, or for identification of a precancerous or cancerous lesion may include, for example: compounds described in Ferrante et al., Cancer Chemother Pharmacol 43 Suppl: S61-8 (1999) may be used by local delivery to the breast duct, including e.g., paclitaxel; MUC1-KLH plus QS-21 as described in Adluri et al., Br J Cancer 79(11-12): 1806-12 (1999); targeting molecules described in Tagliabue et al., Eur J Cancer 34(12): 1982-3 (1998); immunotoxins described in Lorimer et al., Clin Cancer Res 1(8): 859-64 (1995); antihuman endoglin immunotoxin as described in Seon et al., Clin Cancer Res 3(7): 1031-44 (1997); a synthetic MUC1 peptide as described in Reddish et al., Int J Cancer 76(6): 817-23 (1998); anti-HER-2 immunoliposomes as described in Park et al., Cancer Lett 118(2): 153-60 (1997)and Park et al., Proc Nat'l Acad Sci 92(5): 1327-31 (1995); bispecific antibodies such as the one described in Valerius et al., Blood 90(11): 4485-92 (1997); antibody BrE-3 murine IgG1 monoclonal antibody as described in DeNardo et al., J Nucl Med 38(8): 1180-5 (1997); peptide vaccines as described in Moscatello et al., Cancer Res 57(8): 1419-24 (1997); MUC1 monoclonal antibodies as described in Peterson et al., Cancer Res 55(23 Suppl): 5847s-5851s (1995); monoclonal antibodies as described in Howell et al., Int J Biol Markers 10(3): 129-35 (1995); and molecules that target the L6 antigen as described, e.g., in Marken et al., J Biol Chem 269(10): 7397-401 (1994).

Some antibody targeting molecules that can be used to identify and/or treat a premalignant or malignant cancer lesion (e.g., precancer or cancer) include antibodies specific for 44-3A6 antigen (see Duda et al., Tumor Biol 12: 254-260 (1991)), A-80 antigen (see Eriksson et al., Hum Pathol 23(12): 1366-1372 (1992); Shin et al., APMIS 97: 1053-1067 (1989); Shin et al., APMIS 97(12): 1053-67 (1989)), DF3 antigen (see Ohuchi et al., JNCI 79(1): 109-(1987)), H23 antigen (see Zaretsky et al., FEBS 265: 1,2 46-50; Kedyar et al., Proc Nat'l Acad Sci 86(4): 1362-6 (1989); Stein et al., Int J. Cancer 47(2): 163-9 (1991)), 83 D4 antigen (see Pancino et al., Hybridoma 9(4): 389- (1990); Konska et al., Int J Oncol 12(2): 361-7 (1998); Pancino et al., Br. J. Cancer 63(3): 390-8 (1991)), and JDB1 antigen (see Strelkauskas and Taylor, Cancer Immunol Immunother 23(1): 31-40 (1986) and Strelkauskas et al., Hum Antibodies Hybridomas 5(3-4): 157-64)); antibody B72.3 (see Tavassoli et al., Am J Surg Pathol 14(2): 128-33 (1990), Prey et al., Hum Pathol 22(6): 598-602 (1991), Lamki et al., J Nucl Med 32(7): 1326-32 (1991), and Contegiacomo et al., Eur J Cancer 30A(6): 813-20 (1994)); antibody 323/A3 as described in Courtney et al., Br J Cancer Suppl 10: 92-5 (1990); and carcinoembryonic antigen (CEA) as described in Kuhajda et al., Cancer 52: 1257-64 (1983). Monoclonal antibodies related to breast cancer in general and some specific monoclonal antibodies related to breast cancer are discussed in Thor 13(4): 393-401 (1986).

Some preferred agents are antibodies, or fragments of antibodies as described above, and also preferred are small molecules that the body can readily absorb and which can be conjugated to an imaging or therapeutic molecule. These small molecules and/or antibodies and imaging agents can include, in their conjugated form, such combinations as for example Technetium-99 m Sestamibi, ¹⁸F-FDG, Gadolinium-DOTA, and Fab portion of anti-CEA (CEA-Scan) conjugated to an imaging or therapeutic molecule. 99 m Technetium (99 m-Tc) conjugated to the small molecule sestamibi is described in a systemic delivery context in Obwegeser et al, J. Nucl. Med 2000, 41(3):426-8 and Obwegeser et al, Eur. J. Nucl Med. 1999, 26(12): 1553-9. Use of 14C-Gadolinium-tetraazacyclododecane-tetraacetic acid (Gd-DOTA) and Gadolinium-diethylenetriaminepentaacetic acid (Gd-DTPA) for cancer imaging by systemic administration is described in Takeda et al, Eur. J. Radiol. 1998, 26(3);290-6, Curtlet et al. Invest. Radiol 1998, 33(10):752-61, and Gd-DOTA is further described in Loubeyre et al. Magn. Reson. Imaging 1999 17(4):627-31. Breast cancer imaging using (18)F-Fluorodeoxyglucose (FDG) and positron emission tomography (PET) is described in Schelling et al, J. Clin. Oncol. 2000 18(8):1689-1695. Breast cancer imaging with systemically administered radiolabeled antibodies is described in Goldenberg and Nabi, Semin. Nuci. Med. 1999, 29(1):41-8 using CEA antibody Fab′ fragment approved for colorectal cancer detection, Arcitumomab (also called CEA-Scan available from Immunomedics, Morris Plains, N.J.).

In addition to the methods described above, the present invention also includes systems and kits for cannulation individual ductal networks in a breast and for delivering diagnostic and/or therapeutic agents to the ductal network. The systems will include catheters configured to access individual ductal networks, usually via an orifice in the nipple of the breast. Suitable catheters for providing such access are described in co-pending U.S. Pat. No. 09/301,058, the full disclosure of which is incorporated herein by reference. The systems will further include at least one labeling or therapeutic reagent, as described above, usually present in a vial or other sterile container in an amount suitable for performing a procedure on a patient, usually referred to as a “unit dose.” The system may include other components as well, such as those present in the kits described below.

Kits will comprise at least an access catheter in combination with instructions setting forth any of the diagnostic and/or therapeutic methods of the present invention. In addition, the kits may comprise any reagent(s) necessary to perform the methods and will usually comprise packaging for holding the catheter(s), instructions for use, and optionally reagents and any other kit components that may be desired.

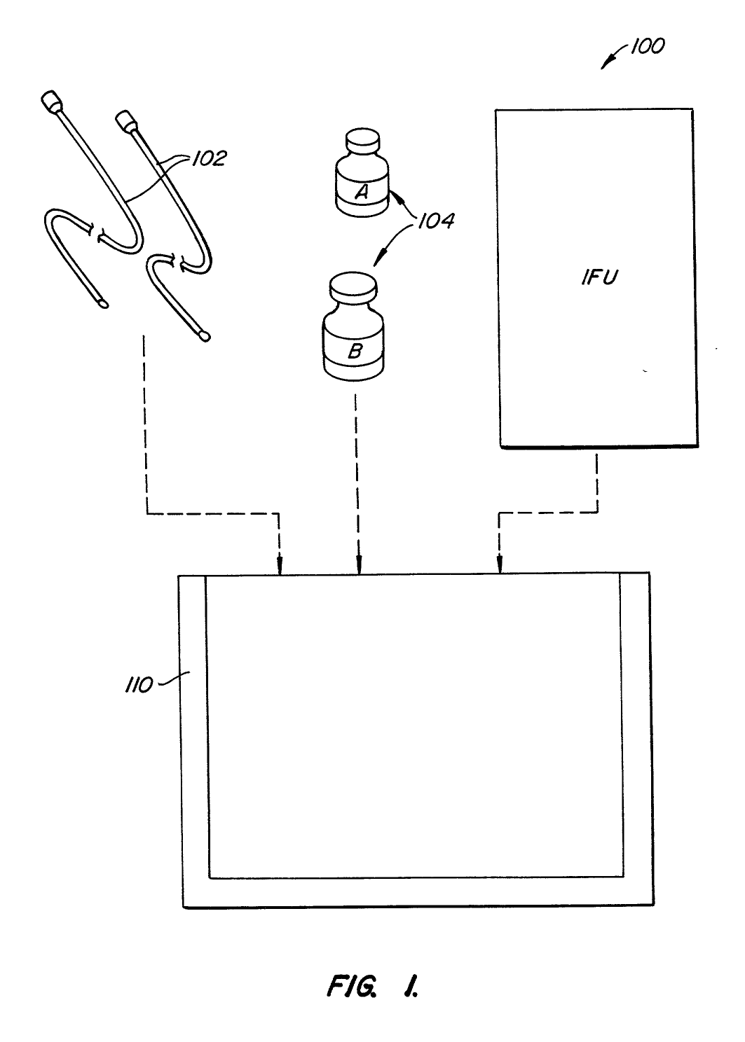

Referring now to FIG. 1, an exemplary kit 100, comprises a pair of access catheters (and optionally more), instructions for use (IFU), and reagents in vials 104. The instructions for use will usually be printed on a separate paper or in a separate booklet, although all or part of the instructions may be provided on the packaging or elsewhere. The packaging 110 may comprise a box, bag, tray, tube, pouch, or other conventional medical device package. Use of at least the access catheters 102 will be maintained sterile within the package. Systems may comprise the catheter(s) 102 and reagent(s) 104, optionally with other components.

Below are provided examples of the invention which are not directed to be limiting, but rather illustrative of the invention.

### EXAMPLES

### 1. In vivo Tumor Localization of Breast Cancer Cells and Treatment—SCID Mice

Young postpartum female SCID mice are injected with breast cancer cells such as BT-474 or MCF7 cells into their breast ducts, and subcutaneous implants of estrogen pellets to support the tumorigenic growth of these cells. After a few days to two weeks, the breasts ducts of these mice are accessed with a fine single lumen catheter to infuse saline, squeeze the breast and collect the saline mixed with ductal fluid to determine the presence of human breast cancer cells by cytological analysis of the retrieved cells.

The mice who are found to harbor human breast cancer cells are divided into two groups. The first group is mice who do not contain palpable tumors and who are mammographically negative, the second group is mice who contain palpable tumors. Anti-p185^{Her-2} immunoliposomes (described in WO 97/38731) containing image contrast enhancement agent such as Gd³⁺.Dy³⁺,Tc and In (described in U.S. Pat No. 5,512,294) are administered to mice from both groups by accessing the breast ducts at the nipples to contact the tumor as described in WO 97/38731. After 30 min to an hour, the accessed breasts are washed with saline solution to remove nonspecifically bound immunoliposomes. An MRI is conducted on the animals to determine the location of breast cancer lesions inside the breast ducts. Information of lesion location is correlated between the MRI, repeated mammograms and physical examination. The linear regression is made between the size of tumor and the MRI signal resonating from the tumor or lesion. The extrapolation of the regression is used to determine the size of tumors or lesions which are undetected by mammogram and/or physical exam.

Alternatively, other imaging agents including radioactive imaging agents such as 125-iodine and 131-iodine and 111-Indium can be used instead of immunoliposomes. Gamma counter camera is used for imaging in that context if those agents are used.

A subsequent treatment experiment is conducted with a subset of the mice having human cancer. Her-2 antibody conjugated liposomes are used to deliver yttrium-90 to the cancer cells. The breast ducts having cancer are later infused with saline to collect the ductal cells and look for abnormality. If abnormality persists, another treatment is delivered, and the condition monitored.

### 2. In vivo Tumor Localization of Breast Cancer Cells and Treatment—Transgenic Rats

Several young post-partum c-erbB-2 transgenic female rats (Davies BR et al., 1999, Am J Pathol 155: 303) are used for this study. After their first pregnancy, the breasts of these rats are accessed with a fine single lumen catheter to infuse saline, squeeze the breast and collect saline mixed with ductal fluid to determine the presence of atypical cells or carcinoma by cytological analysis of the retrieved cells.

The rats who are found to harbor human breast cancer cells are divided into two groups. The first group is rats who do not contain palpable tumors and mammographically negative; the second group is rats who contain palpable tumors. Anti-p185^{Her2} immunoliposomes (described in WO 97/38731) containing image contrast enhancement agent such as Gd³⁺, Dy³⁺, Tc and In (described in U.S. Pat. No. 5,512,294) are administered to rats from both groups by accessing the breast ducts at the nipples to contact the tumor described in WO 97/38731. After 30 min to an hour, the accessed breasts are washed with saline solution to remove nonspecifically bound immunoliposomes. An MRI is conducted on the animals to determine the location of breast cancer cells inside their breast ducts. The correlation of tumor location is determined between the MRI and repeated physical examination or mammogram. A linear regression is made between the size of tumor and the MRI signal resonating from the tumor or lesion. An extrapolation of the regression is used to determine the size of tumors undetected by mammogram and/or physical exam.

Alternatively, other imaging agents including radioactive imaging agents such as 125-iodine and 131-iodine and 111-Indium can be used instead of immunoliposomes. Gamma counter camera would be used for such imaging.

### 3. FITC Conjugated F(ab′)₂. Fragments are Cleared from Mammalian Breast Ducts

An experiment was conducted to determine that FITC conjugated antibody can clear from a rabbit breast duct within about 27 hours or less. A female rabbit was acquired from Kralek farms (Santa Cruz, Calif.). The amount of anesthesia required for the rabbit was calculated based on the weight (5 kg). A cocktail of ketamine and xylane is administered; Ketamine is administered at 50 mg/kg and xylane at 3 mg/kg. For a 5 kg rabbit then 250 mg of ketamine and 15 mg of xylane was used. Appropriate dilutions were prepared. The anesthetized rabbit was shaved and the nipple surface treated with keratin removing agent. FITC conjugated CEA F(ab′)₂ fragments (FITC is fluorescein isothiocyanate isomer) available from Dako(Denmark DK 2600 Glostrup) were used. The FITC conjugated F(ab′)₂ fragment of swine anti-rabbit immunoglobins (code no. F 0054) were presented as 0.5 ml of FITC conjugated F(ab′)₂ CEA antibody at a concentration of 0.2 g/L of antibody diluted from the original concentration of 0.5 g/L with 1.5 ml of 0.1% BSA. Two concentrations of FITC-F(ab′)₂ were used: 20 ug/ml and 50 ug/ml. FITC-Fab is prepared in PBS solution containing 500 ug/ml BSA. Isosofan blue is mixed with the FITC-Fab solution as a marker for marking injected ductal area on the skin for each breast. The positive control is obtained through injection of 0.5 ml FITC-Fab into a nipple at a concentration of 20 ug/ml of FITC and another nipple at a concentration of 50 ug/ml of FITC. Positive controls were injected into the nipples after the rabbit was sacrificed but before harvesting to show the absolute maximum of fluorescence possible when none of the antibody is cleared. After the antibody mixture with label was allowed a 30 minute incubation in a reaction vial, 0.5 ml of the FITC-F(ab′)₂ mixture with isosofan blue (to visually follow the injection) was injected into each nipple. Injections were staggered so that all time points finished at the same harvest time. Thus, the nipples scheduled for a 27 hour incubation were injected on day 1, and harvested some 27 hours later on day 2; and nipples scheduled for a 2 hour incubation were injected on day 2, 2 hours before harvesting. Both the 27 hour and 2 hour incubations were “chased” twice at half hour intervals by 0.5 ml PBS. At the time of harvesting the rabbit was sacrificed, and before cutting or dissection of the nipples, isofan blue dye was injected to identify the location of the ducts that were injected with the conjugate. After sacrificing the animal, but before harvesting the positive control nipples were injected. The results were determined by taking a picture of the fluorescence from the back of the breast tissue, and visually approximating the intensity differences of the fluorescence. See the table 1 below for the results.

TABLE 1

| | | | Ab | | | Duration | | |
|---|---|---|---|---|---|---|---|---|
| | | [Ab] | injection | | | of Ab | Harvest | |
| Rabbit | Nipple | ug/ml | time | 1^{st} PBS | 2^{nd} PBS | incub. | time | Results |
| A | #3 | 20 | 12:10 p | 12:40 p | 2:10 p | 27 hours | 3:00 p | No fluor. detected |
| | | | day 1 | day 1 | day 1 | | day 2 | |
| A | #4 | 50 | 12:10 p | 12:40 p | 12:40 p | 27 hours | 3:00 p | No fluor. detected |
| | | | day 1 | day 1 | day 1 | | day 2 | |
| A | #5 | 20 | 12:50 p | 1:20 p | 2:50 p | 2 hours | 3:00 p | 10% of positive |
| | | | day 2 | day 2 | day 2 | | day 2 | control |
| A | #6 | 50 | 12:50 p | 1:20 p | 2:50 p | 2 hours | 3:00 p | 10% of positive |
| | | | day 2 | day 2 | day 2 | | day 2 | control |
| A | #1 | 20 | 4:00 p | no | no | 0 hours | 3:00 p | Positive reference |
| | | | day 2 | | | | day 2 | |
| A | #2 | 50 | 4:00 p | no | no | 0 hours | 3:00 p | Positive reference |
| | | | day 2 | | | | day 2 | |
| A | #7 | 20 | no | | | | 3:00 p | No signal |
| | | | | | | | day 2 | |
| A | #8 | 50 | no | | | | 3:00 p | No signal |
| | | | | | | | day 2 | |

### 4. Effects of Blocking and Chasing on Antibody Clearance

A rabbit was prepared as described in experiment 3 above. Six nipples on the rabbits were marked and cleaned. The anesthetized rabbit was shaved and the nipple surface treated with keratin removing agent. FITC conjugated CEA F(ab′)₂ fragments (FITC is fluorescein isothiocyanate isomer) available from Dako(Denmark DK 2600 Glostrup) were used. The FITC conjugated F(ab′)₂ fragment of swine anti-rabbit immunoglobins (code no. F 0054) were presented as 0.5 ml of FITC conjugated F(ab′)₂ CEA antibody at a concentration of 0.2 g/L of antibody diluted from the original concentration of 0.5 g/L with 1.5 ml of 0.1% BSA. A concentration of FITC-F(ab′)₂ at 50 ug/ml was used for injection into the rabbit nipples. FITC-Fab was prepared in PBS solution containing 500 ug/ml BSA. Isosofan blue is mixed with the FITC-Fab solution as a marker for marking injected ductal area on the skin for each breast. Positive controls were injected into the nipples after the rabbit was sacrificed but before cutting the tissue to show the absolute maximum of fluorescence possible when none of the antibody is cleared. After the antibody mixture with label was allowed a 30 minute incubation in a reaction vial, 0.5 ml of the FITC-F(ab′)₂ mixture with isosofan blue (to visually follow the injection) was injected into each nipple. Injections were staggered so that all time points finished at the same harvest time. Thus, the nipples scheduled for a 20 hour incubation were injected on day 1, and harvested some 20 hours later on day 2; and nipples scheduled for a 2 hour incubation were injected on day 2, 1.5 hours before harvesting. Two nipples (one designated for a 20 and one for a 2 hour injection) were chased once with 0.5 ml PBS. Two nipples (one each at 20 hour and 2 hour incubation periods with the antibody-FITC solution) were selected for blocking before antibody injection with 2.5 ml of 0.5 mg/ml BSA to block proteins which otherwise might bind antibody nonspecifically and skew the FITC reading. The positive control is obtained through injection of 0.5 ml FITC-Fab into a nipple at a concentration of 50 ug/ml of FITC. Thus positive controls were at 0 hours of incubation and done at two different concentrations of BSA in the antibody solution (at 0.5 mg/ml and 1 mg/ml). At the time of harvesting the rabbit was sacrificed, and before cutting or dissection of the nipples, isofan blue dye was injected to identify the location of the ducts that were injected with the conjugate. After sacrificing the animal, but before cutting tissue the positive control nipples were injected with antibody and BSA in a single solution. The results were determined by taking a picture of the fluorescence from the back of the breast tissue, and visually approximating the intensity differences of the fluorescence. See the table 2 below for the results.

TABLE 2

| | Ab | | | | |
|---|---|---|---|---|---|
| Nipple | incub. | Blocking | PBS | [BSA] | Results |
| 1 | 0 hours | No | No | 0.5 mg/ml | Positive control (10×) |
| 2 | 0 | No | No | 1 mg/ml | Positive control (10×) |
| 3 | 2 | No | No | 0.5 mg/ml | 0.5× antibody |
| | | | | | remaining |
| 4 | 20 | No | No | 1 mg/ml | 0.3× antibody |
| | | | | | remaining |
| 5 | 2 | Yes | No | 0.5 mg/ml | 0.3× antibody |
| | | | | | remaining |
| 6 | 20 | Yes | No | 1 mg/ml | 0.3× antibody |
| | | | | | remaining |
| 7 | 2 | No | Yes | 0.5 mg/ml | 0.4× antibody |
| | | | | | remaining |
| 8 | 20 | No | Yes | 1 mg/ml | 0.3× antibody |
| | | | | | remaining |

The results of Table 2 indicate that there is no difference in the amount of fluorescence remaining for the 20 hour groups, regardless of whether blocking, or PBS washing were employed in the procedure. Among the 2 hour groups, the fastest cleared was the group with blocking (nipple 5) which appeared to clear as much as the 20 hour group, and better than nipple 3, and nipple 7 which were both 2 hour incubations; nipple 3 was without blocking or PBS washing, and nipple 7 was with PBS washing but without blocking. All results indicating remaining fluorescence were based on a relative 10X of the positive control, and were qualitative evaluations of the remaining fluorescence. The results indicate a satisfactory clearance rate of conjugated antibody at 2 hours of antibody incubation in the animal tested.

### 5. Imaging in Tumor-Bearing Rats using 99m-Tc-sestamibi, (14C)-Gd-DOTA, Gd-DPTA, (18F)FDG, and 99m-Tc-CEA-Scan

Breast cancer is induced in rats by administration of 1-methyl-1-nitrosourea (MNU). Rats who develop palpable mammary tumors are selected. Two rats are selected for injection of each of the following reagents: 99m-Tc-sestamibi, (14C)-Gd-DOTA, Gd-DPTA, (18F)FDG, and 99m-Tc-CEA-Scan for a total of 10 rats for an initial study. The mammary gland bearing the tumor of each rat is administered by cannulation a solution volume of about 100 ul PBS as follows:

99m-Tc-sestamibi (5.7-6.8 uCi/ml in PBS)

viewing after 2 min, 5 min, 10 min, 20 min, 40 min, 80 min, and 160 min. incubation.

(14C)-Gd-DOTA (2.9 mmol/ml)

viewing after 1 min, 2 min, 4 min, 7 min and 15 min. incubation.

Gd-DPTA (2.9 mmol/ml)

viewing at 1 min, 2 min, 4 min, 7 min and 15 min. incubation.

(18F)FDG (10 mCi)

viewing at 30 min, 60 min, and 120 min. and incubation.

99m-Tc-CEA-Scan (5.7-6.8 uCi/ml in PBS)

viewing at 5 min, 15 min, 30 min, 60 min, 120 min, 4 hours, 8 hours and 16 hours, incubation.

Comparisons are made for each of the two rats administered the same agent, and between all rats each pair of which is administered a different agent. Conclusions about dosages and advantages or disadvantages of particular agents can be made from these data. Clearance of the agents including sestamibi and Fab′ CEA are expected due to the smaller size of these molecules and their presumed ability to pass through the ductal epithelial lining and be processed and cleared by the body, although the invention is not limited to theories upon how it works.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method of identifying the location of premalignant or malignant breast cancer within a mammalian body comprising a breast duct or breast ductal network, said method comprising:
providing a coupled compound comprising a targeting molecule coupled to an identifying agent;
delivering the coupled compound through a preselected individual breast duct in an amount sufficient to identify premalignant or malignant cancerous cells;
identifying any bound cells; and
allowing any unbound coupled compound to clear in the body;
wherein any coupled compound that does not bind a cell in the duct diffuses out of the breast ductal network and is absorbed and cleared in the body without requiring removal by a practitioner of the unbound coupled compound.

2. A method as in claim 1, wherein delivering comprises cannulation or catheterization of the breast duct.

3. A method as in claim 1, wherein the coupled compound is delivered to more than one duct on a breast.

4. A method as in claim 1, wherein the cells are identified for the purpose of excising tissue surrounding and including the cells.

5. A method as in claim 1, wherein the targeting agent comprises an agent selected from the group consisting of a protein, a polypeptide, a peptide, an antibody, an antibody fragment, a F(ab′) fragment of an antibody, a F(ab′)₂ fragment of an antibody, an Fc portion of an antibody, a heavy chain of an antibody, a light chain of an antibody, a humanized antibody, a humanized antibody fragment, a ligand, a receptor, a drug, a chemical, a lipid, a liposome, a small molecule, and a nucleic acid.

6. A method as in claim 5, wherein the targeting agent is a small molecule and the small molecule is sestamibi.

7. A method of identifying the location of premalignant or malignant breast cancer within a mammalian body comprising a breast duct or breast ductal network, said method comprising:
providing a identifying agent which binds to premalignant and/or malignant ductal cells; and
delivering the identifying agent through a preselected individual breast duct in an amount sufficient to bind to and identify premalignant or malignant cancerous cells;
identifying any bound cells; and
allowing any unbound identifying agent to clear in the body;
wherein any identifying agent that does not bind a cell in the duct diffuses out of the breast ductal network and is absorbed and cleared by the body without requiring removal by a practitioner of the unbound identifying agent.

8. A method as in claim 7, wherein delivering comprises cannulation or catheterization of the breast duct.

9. A method as in claim 7, wherein the identifying agent is delivered to more than one duct on a breast.

10. A method as in claim 7, wherein the cells are identified for the purpose of excising tissue surrounding and including the cells.

11. A method as in claim 7, wherein the identifying agent comprises an agent selected from the group consisting of a protein, a polypeptide, a peptide, an antibody, an antibody fragment, a F(ab′) fragment of an antibody, a F(ab′)₂ fragment of an antibody, an Fc portion of an antibody, a heavy chain of an antibody, a light chain of an antibody, a humanized antibody, a humanized antibody fragment, a ligand, a receptor, a drug, a chemical, a lipid, a liposome, a small molecule, and a nucleic acid.

12. A method of treating premalignant or malignant breast cancer located within a mammalian body, said method comprising:
providing a coupled compound comprising targeting molecule coupled to a therapeutic agent; and
delivering the coupled compound through a preselected individual breast duct in an amount sufficient to inhibit proliferation of the cancerous cells; and
allowing unbound coupled compound to clear through the body; wherein any coupled compound that does not bind a cell in the duct diffuses out of the breast ductal network and is absorbed and cleared by the body without requiring removal by a practitioner of the unbound coupled compound.

13. A method as in claim 12, wherein delivering comprises cannulation or catheterization of the breast duct.

14. A method as in claim 12, wherein the coupled compound is delivered to more than one duct on a breast.

15. A method as in claim 12, wherein the targeting agent comprises an agent selected from the group consisting of a protein, a polypeptide, a peptide, an antibody, an antibody fragment, a F(ab′) fragment of an antibody, a F(ab′)₂ fragment of an antibody, an Fc portion of an antibody, a heavy chain of an antibody, a light chain of an antibody, a humanized antibody, a humanized antibody fragment, a ligand, a receptor, a drug, a chemical, a lipid, a liposome, a small molecule, and a nucleic acid.

16. A method as in claim 15, wherein the targeting agent comprises a small molecule and the small molecule is sestamibi.

17. A method as in claim 12, wherein the therapeutic agent is selected from the group consisting of a cytotoxic agent, a cytolytic agent, a cytostatic agent, a growth inhibiting agent, an antagonist, an agonist, and a drug or agent containing liposome.

18. A method as in claim 12, wherein the therapeutic agent comprises an agent with therapeutic activity against cancerous or precancerous cells that can be coupled to a targeting agent.

19. A method of treating a premalignant or malignant breast cancer located within a mammalian body, said method comprising:
providing a targeting molecule itself having therapeutic activity; and
delivering the targeting molecule through a preselected individual breast duct in an amount sufficient to inhibit proliferation of the premalignant or malignant cells, wherein at least a portion of the targeting molecule binds to premalignant or malignant cells; and
allowing unbound targeting molecule to clear through the body;
wherein any targeting molecule that does not bind a cell in the duct diffuses out of the breast ductal network and is absorbed and cleared by the body without requiring removal by a practitioner of the targeting molecule.

20. A method as in claim 19, wherein delivering comprises cannulation or catheterization of the breast duct.

21. A method as in claim 19, wherein the targeting molecule is delivered to more than one duct on a breast.

22. A method as in claim 19, wherein the targeting molecule comprises an agent selected from the group consisting of a protein, a polypeptide, a peptide, an antibody, an antibody fragment, a F(ab′) fragment of an antibody, a F(ab′)₂ fragment of an antibody, an Fc portion of an antibody, a heavy chain of an antibody, a light chain of an antibody, a humanized antibody, a humanized antibody fragment, a ligand, a receptor, a drug, a chemical, a lipid, a liposome, a small molecule, and a nucleic acid.

23. A method as in claim 22, wherein the targeting molecule comprises a small molecule, and the small molecule is sestamibi.

24. A method as in claim 19, wherein the therapeutic activity is selected from the group consisting of a cytotoxicity, a cytolytic activity, cytostatic activity, growth inhibition, antagonism, an agonism, and immunotoxicity.

25. A method as in claim 19, wherein the therapeutic activity is effective against cancerous or precancerous cells.

26. A method as in claim 12 or 19, wherein the premalignant or malignant breast cancer comprises cells having a stage selected from the group consisting of hyperplasia, atypical hyperplasia, low-grade ductal carcinoma in situ, high-grade ductal carcinoma in situ, and invasive carcinoma.

27. A kit for localizing or treating lesions in a breast duct, said kits comprising:
at least one catheter configured to access a ductal network in a human breast; and
instructions for use setting forth a method according to any of claims 1 to 26.

28. A kit as in claim 27, further comprising at least one container holding a reagent which is used in the method being performed with the kit.

29. A kit as in claim 27, further comprising a package holding the catheter and the instructions for use.

30. A kit as in claim 27, further comprising a reagent, wherein the reagent comprises an agent comprising at least a portion selected from the group consisting of a protein, a polypeptide, a peptide, an antibody, an antibody fragment, a F(ab′) fragment of an antibody, a F(ab′)₂ fragment of an antibody, an Fc portion of an antibody, a heavy chain of an antibody, a light chain of an antibody, a humanized antibody, a humanized antibody fragment, a ligand, a receptor, a drug, a chemical, a lipid, a liposome, a small molecule, and a nucleic acid.
